(19) United States
(12) Patent Application Publication
(10) Pub. No.: US 2006/0233893 A1
(43) Pub. Date: Oct. 19, 2006

(54) Aqueous solutions for the reduction of fatty tissue

(76) Inventors: Serge Bernstein, Epinay Sur Seine (FR); Yves Crassas, Lyon (FR)

Correspondence Address: STITES & HARBISON PLLC, 1199 NORTH FAIRFAX STREET, SUITE 900, ALEXANDRIA, VA, 22314, US

(21) Appl. No.: 11/259,757

(22) Filed: Oct. 27, 2005

(30) Foreign Application Priority Data
Oct. 27, 2004 (FR) 04 11474

(51) Int. Cl. A61K 33/00; A61K 31/685; A61K 31/24; A61K 31/137

(52) U.S. Cl. 424/722; 514/78; 514/537; 514/649

(57) ABSTRACT

The present invention relates to isotonic or hypotonic aqueous solutions which are hyperkalaemic or which contain tiratricol and/or phosphatidylcholine, and to their use in the reduction of fatty tissue.

The present invention relates to aqueous solutions that can be used to reduce fatty tissue, in particular to an isotonic or hypotonic solution which is hyperkalaemic or which contains tiratricol and/or an amphoteric compound comprising a lipophilic pole and a hydrophilic pole, such as phosphatidylcholine, and also to their use in the reduction of fatty tissue, in particular in a method for the aesthetic treatment of cellulite by lipotomy.

In the context of the present invention, the term “cellulite” denotes, according to common parlance, aesthetic cellulite, that is to say, a generally very localised hypertrophy of the adipose tissue forming bumps called “orange peel skin”.

Lipotomy, or hypo-osmolar treatment, is a technique which enables cellulite to be reduced and which involves intra-cellular hyperhydration, membrane weakening or potassium tonicity of adipocytes in order to permit their destruction. That technique is based on the principle of diluting the interstitial liquid in which the adipocytes lie in order to induce differences in osmotic pressure on each side of the adipocyte membrane. Those differences in osmotic pressure result in a flow of liquid from the compartment of lower osmolarity (the interstitial liquid) towards the compartment of higher osmolarity (the cytoplasm of the adipocytes). The technique was initially developed in France by Dr Serge Bernstein (French patent application FR 97 14073) and in the United States by Doctor Steven Hoefflin (Aesthetic Surgery, 2002, 22(6)).

The osmole (Osm) is a physical unit of measurement of osmotic pressure expressed in moles per litre. When one mole of a substance is dissolved in one litre of water, it exerts an osmotic pressure of one osmole. The osmolarity is the concentration of dissolved substance exerting an osmotic power. The osmolarity of plasma is regarded as being from 280 mOsm/l to 330 mOsm/l. The osmolarity of cell cytoplasm is 394 mOsm/l.

The terms “hypotonia”, “isotonia” and “hypertonia” are used here to denote a compartment whose osmolarity is lower than, equal to and higher than, respectively, the osmolarity of plasma.

The lipotomy technique conventionally used comprises the intra-fatty administration of a so-called “destruction” solution, which is highly hypotonic (having an osmolarity of generally from 120 mOsm/l to 20 mOsm/l) and which brings about hyperhydration of adipocytes. The increase in volume of the adipocytes brings about a weakening of their membrane, and even cell lesions. The destruction of the adipocytes is completed by slight mechanical traumatism, for example, by the application of a transcutaneous ultrasound field or by massage of the palpation-rolling type in particular. The ultrasound generates a partial vacuum which brings about the “explosion” of the cells by cavitation. The lesion and then destruction of the adipocytes leads to a release of fats as well as triglycerides, free fatty acids and other cell contents in the plasma before they are degraded by the liver in order to be eliminated. The cell debris is removed by the lymphatic system.

However, that destruction step enables only a limited volume of fatty tissue to be treated. Beyond one to two centimetres' thickness of fatty tissue, not all of the adipocytes can be targeted. The repetition of the destruction step is problematic because the use of a solution of very low osmolarity brings about the establishment of tissue fibrosis which requires approximately 2 to 3 months to be absorbed. Since a highly hypotonic solution cannot be administered again before the fibrosis is absorbed, the destruction step, if it had to be repeated, would lead to an aesthetic treatment by lipotomy of far too long a duration. In addition, a recurrence is often observed 2 to 3 months after the destruction step. Treatments spaced apart to that extent would therefore impair the overall efficacy of the method.

Recurrence is also prevented using a so-called “follow-up” treatment which comprises up to three administrations, at intervals of approximately one month, of a solution which is only slightly hypotonic (approximately 200 mOsm/l) and which enables the proteins trapped in the fibrous tissue to be freed and thus exerts a draining effect.

In the case of persons having too large a volume of fat for the aesthetic treatment method by lipotomy to be entirely efficacious, the treatment is combined with liposuction, which eliminates both the emulsified fats that are to be absorbed and any adipocytes that have not been destroyed. Liposuction is, however, an invasive technique which is more traumatizing than lipotomy and it is therefore preferable to be able to dispense with it.

The object of the present invention is therefore to improve the currently used methods of aesthetic treatment by lipotomy in such a manner as to avoid repetition of the administration of highly hypotonic solutions and/or having to resort to complementary liposuction, when the volume of fat to be eliminated is too large. A volume of fat is regarded as being too large when the thickness of the fatty tissue is determined by echography to be greater than one or two centimetres. It will be appreciated that the improvements afforded by the present invention are also useful when the volume of fat is lower, since they help to increase the efficacy of lipotomy.

The inventors have thus developed a hyperkalaemic iso- or hypotonic solution whose administration in the course of a method of aesthetic treatment by lipotomy, as a preliminary to the destruction phase, enables the volume of the adipocytes to be reduced and thus a maximum of cells to be targeted at the same time in the course of the destruction step. Since the administration of that solution can be repeated at short intervals, it enables the overall duration of the lipotomy method to be reduced. Furthermore, owing to the use of that solution, the volume of fat treated during the destruction phase is smaller, and it is therefore no longer necessary to resort to liposuction.

The inventors have additionally demonstrated that a synergy of action may also be observed between a hyperkalaemic isotonic solution, or an optionally hyperkalaemic hypotonic solution, or and some substances, such as tiratricol (triiodothyroacetic acid) or an amphoteric compound, such as phosphatidylcholine.

Hyperkalaemic Iso- or Hypotonic Solution

The inventors have developed a hyperkalaemic hypotonic or isotonic solution which, when necessary, permits a massive reduction in fatty tissue up to a thickness of 1 to 2 cm, which is the thickness on the basis of which a treatment by a destruction solution can be used with sufficient efficacy not to have to be repeated.

Potassium is a cation which has a substantially intracellular distribution (98% against 2% in the extracellular medium). The kalaemia, that is to say, the concentration of potassium in the plasma, is approximately 4 mmol/l, which corresponds to an osmolarity associated with potassium of 4 mOsm/l. The intracellular osmolarity associated with potassium in the adipocytes is approximately 160 mOsm/l. The maintenance of this unequal distribution on each side of the plasma membranes is ensured thanks to active transport via Na, K and ATP pumps. In the context of the Application, a “hyperkalaemic solution” denotes a solution in which the potassium concentration is higher than that of the plasma; preferably, it is a solution comprising from 20 to 110 mOsm/l, and even more preferably comprising from 25 to 110 mOsm/l of potassium.

Owing to that active transport, the potassium added to a hypotonic solution would be transported and collected in the adipocytes, thus helping to enhance the osmotic pressure gradient between the interstitial medium, diluted by the hypotonic solution, and the intracellular medium. Potassium is also cytotoxic for the cells when it has a high concentration. A K/Na ratio greater than 1 increases efficacy.

However, the use of a potassium concentration sufficiently high to observe an increased reducing effect on fatty tissue in a hypotonic solution, which is efficacious in respect of lipotomy, is not possible because the osmolarity of the solutions used is so low (the destruction solutions have an osmolarity of from 60 to 90 mOsm/l) that the enhancement of the destructive effect by potassium could lead to accidents, with the risk of fatty and cutaneous necrosis requiring surgical exeresis. The inventors have found that, compared with a destruction solution having an osmolarity of 60 mOsm/l, the association of potassium with a destruction solution having a higher osmolarity does not offer any advantage in terms of tissue recovery and, for all that, does not reduce the risk of necrosis and fibrosis.

The inventors have demonstrated that a real gain in terms of tissue recovery permitting repeated treatments without risk is observed only as of an osmolarity of 167 mOsm/l, for hyperkalaemic solutions. The same hypotonic solutions without potassium do not exhibit efficacy in terms of fat reduction.

The inventors have developed a moderately hypotonic or isotonic solution (having an osmolarity of from 167 to 330 mOsm/l) containing potassium at a level of approximately from 20 to 110 mOsm/l, optionally associated with another agent whose intracellular penetration is facilitated by the increase in the membrane porosity due to the hyperhydration and the increase of the cell volume. This administration constitutes a preparatory step for lipotomy, when the volume of fat to be eliminated is too large, that is to say, greater than 1 to 2 cm thickness as determined by echography.

The invention proposes more particularly a hypotonic aqueous solution having an osmolarity of from 167 mOsm/l to 257 mOsm/l and comprising from 20 to 110 mOsm/l of potassium. In the context of this Application, this solution is called the “preparative solution”.

Preferably, the osmolarity of the solution is from 227 to 257 mOsm/l.

Preferably, the osmolarity of the potassium in the solution is from 25 to 110 mOsm/l, preferably from 28 to 107 mOsm/l, preferably from 40 to 70 mOsm/l, even more preferably from 50 to 60 mOsm/l. According to a preferred embodiment, the solution contains 53.6 mOsm/l of potassium.

Since the solutions used in lipotomy are injected directly into the fatty tissue, in order to prevent them from diffusing and therefore to prevent the dilution effect from being reduced, a vasoconstrictive compound, generally adrenaline, is administered beforehand or at the same time in order to isolate the fatty tissue vascularly. Advantageously, the vasoconstrictor may be diluted directly in the preparative solution according to the invention. The choice of an appropriate concentration is within the competence of the person skilled in the art. For example, the preparative solution according to the invention may also comprise 1.2 mOsm/l of adrenaline.

In the same manner, an anaesthetizing agent may be administered before or at the same time as the preparative solution. Examples of anaesthetizing agents used in lipotomy include lidocaine or procaine. Preferably, the anaesthetizing agent is diluted directly in the preparative solution, for example, with an osmolarity of 13.2 mOsm/l.

Moreover, the preparative solution according to the invention may comprise sodium chloride and/or bicarbonate, which is conventionally used to counterbalance the acidosis which may result from the lesion or the destruction of the adipocytes. The addition of calcium to the solute potentiates the destructive effects.

Thus, according to a preferred embodiment, the preparative solution according to the invention has an osmolarity of from 167 mOsm/l to 257 mOsm/l, preferably from 227 to 257 mOsm/l, and comprises from 50 to 60 mOsm/l of potassium, and optionally 13.2 mOsm/l of xylocaine and/or 1.2 mOsm/l of adrenaline.

For example, it is possible to obtain a solution having an osmolarity of 257 mosm/l and comprising 53.6 mOsm/l of potassium, adding 10 ml of 2% xylocaine (i.e. 3.3 mOsm), 1 ml of adrenaline at 1 mg (i.e. 0.3 mOsm), 10 ml of 10% KCl (i.e. 26.8 mOsm), and 17 ml of 8.4% sodium bicarbonate (i.e. 34 mOsm) in water, for an injectable preparation, in an amount sufficient to make up 250 ml.

The composition may also comprise compounds such as caffeine and/or one or more corticoids.

Since the preparative solution is moderately hypotonic or isotonic, it does not cause tissue damage, in particular necrosis, and the oedema caused by its administration decreases rapidly. This solution may therefore be administered at short time intervals. Although the use of this solution efficiently reduces the thickness of fatty tissue, an early recurrence is nevertheless observed, as of three weeks after treatment, so that this solution cannot in itself represent the only basis of treatment. The use of the solution according to the invention therefore represents a step of preparing fatty tissue before the destruction step, carried out in general by combining the administration of a highly hypo-osmolar solution and by applying slight traumatism (ultrasound, massage).

The invention relates also to the use of an aqueous solution having an osmolarity of from 167 mOsm/l to 330 mOsm/l and comprising from 25 to 110 mOsm/l of potassium, as described above, for the reduction of fatty tissue, in particular for the aesthetic treatment of cellulite.

Iso- or Hypotonic Solution Containing Tiratricol and/or Phosphatidylcholine

The inventors have additionally demonstrated that a synergy of action may also be observed between a hyperkalaemic isotonic solution, or an optionally hyperkalaemic hypotonic solution, and some substances, such as tiratricol (triiodothyroacetic acid) or an amphoteric compound, such as phosphatidylcholine.

The inventors have demonstrated that it was also possible to obtain a reducing effect on fats with an improved tissue recovery time using a hyperkalaemic solution which is isotonic or moderately hypotonic (having an osmolarity of approximately from 120 to 330 mOsm/l) and which contains tiratricol and/or phosphatidylcholine. Such a solution is therefore also appropriate for preparing fatty tissue before destruction by lipotomy.

The inventors have also shown that phosphatidylcholine and/or tiratricol, more specifically phosphatidylcholine, increased the efficacy of a highly hypotonic lipotomy solution (approximately from 20 to 90 mOsm/l) in the destruction of fatty tissue.

The invention therefore relates to a hyperkalaemic isotonic or hypotonic aqueous solution having an osmolarity of from 20 mOsm/l to 330 mOsm/l and comprising from 1000 mg/l to 4800 mg/l of phosphatidylcholine and/or from 13.2 mg/l to 26.4 mg/l of tiratricol. More specifically, the solution may be an aqueous solution having an osmolarity of from 167 mOsm/l to 330 mOsm/l and comprising from 20 to 110 mOsm/l of potassium, as described above, and also comprising from 1000 mg/l to 4800 mg/l of phosphatidylcholine and/or from 13.2 mg/l to 26.4 mg/l of tiratricol.

According to one embodiment, the aqueous solution according to the invention has an osmolarity of from 20 to 90 mOsm/l. According to another embodiment, the aqueous solution according to the invention has an osmolarity of from 120 to 330 mOsm/l.

Preferably, the aqueous solution according to the invention comprises from 1000 mg/l to 1200 mg/l of phosphatidylcholine. Preferably, the aqueous solution according to the invention comprises 13.2 mg/l of tiratricol. Even more preferably, the aqueous solution according to the invention comprises from 1000 to 1200 mg/l of phosphatidylcholine and 13.2 mg/l of tiratricol.

For use in reducing the thickness of fatty tissue before destruction by lipotomy, preference is given to a solution having an osmolarity of from 120 to 330 mOsm/l, comprising from 13.2 mg/l to 26.4 mg/l of tiratricol, preferably 13.2 mg/l, and/or from 1000 mg/l to 4800 mg/l of phosphatidylcholine, preferably from 1000 mg/l to 1200 mg/l.

For use as a solution for destruction by lipotomy, preference is given to a solution having an osmolarity of from 20 to 90 mOsm/l, comprising from 1000 mg/l to 4800 mg/l of phosphatidylcholine, preferably from 1000 mg/l to 1200 mg/l, and/or from 13.2 mg/l to 26.4 mg/l of tiratricol, preferably 13.2 mg/l.

The invention relates also to the use of those solutions in a cellulite treatment by lipotomy.

Method of Aesthetic Treatment By Lipotomy

The invention proposes a method for the aesthetic treatment of cellulite, by lipotomy, which is improved compared with the existing methods. This method enables cellulite to be removed and/or reduced efficiently, in particular when the initial thickness of the fatty tissue is greater than 1 to 2 cm, as determined by echography (reference method). The thickness of the fatty tissue may also be determined by any other appropriate method, such as radiography of the soft tissue or using a scanner.

The method for the aesthetic treatment of cellulite according to the invention comprises the steps that consist in:
a) administering an aqueous solution having an osmolarity of from 167 mOsm/l to 330 mOsm/l and comprising from 20 to 110 mOsm/l of potassium, as defined above, to fatty tissue;
b) optionally repeating step a) until the thickness of the fatty tissue, determined by echography, is less than or equal to 2 cm, especially from 1 to 2 cm;
c) administering an aqueous solution having an osmolarity of from 20 mOsm/l to 90 mOsm/l to the fatty tissue;
d) applying to the said tissue a mechanical treatment capable of bringing about the destruction of the membrane of adipocytes present in the fatty tissue; and
e) optionally administering a solution having an osmolarity of from 180 to 200 mOsm/l to the fatty tissue treated by the mechanical treatment.

Preferably, the hyperkalaemic solution used in step a) is hypotonic, with an osmolarity of from 167 mOsm/l to 257 mOsm/l, preferably from 227 mOsm/l to 257 mOsm/l. The osmolarity of the potassium in this solution is preferably from 25 to 110 mOsm/l, preferably from 40 to 70 mOsm/l, more preferably from 50 to 60 mOsm/l, or even more preferably 53.6 mOsm/l.

The solution used in step a) may also comprise a vasoconstrictor, an anaesthetizing agent, sodium chloride and/or bicarbonate, as described in the present Application in connection with the preparative solution.

According to a preferred embodiment, the solution administered in step a) has an osmolarity of from 167 mOsm/l to 257 mOsm/l, preferably from 227 to 257 mOsm/l, and comprises from 50 to 60 mOsm/l of potassium, and optionally 13.2 mOsm/l of xylocaine and/or 1.2 mOsm/l of adrenaline.

Alternatively, it is possible to use, instead of the hyperkalaemic hypotonic or isotonic solution of step a), a hyperkalaemic isotonic aqueous solution or a hypotonic solution having an osmolarity of from 120 to 330 mOsm/l and comprising from 13.2 mg/l to 26.4 mg/l of tiratricol, preferably 13.2 mg/l, and optionally from 1000 mg/l to 4800 mg/l of phosphatidylcholine, preferably from 1000 mg/l to 1200 mg/l.

Step a) can be repeated as much as necessary, at intervals of from one to two weeks, preferably every week, until the fatty tissue treated has a thickness of from 1 to 2 cm, as determined by echography. This echography is carried out in accordance with standard criteria by measuring the thickness of the hypodermis between the dermis and the muscular aponeurosis.

This step leads to a reduction in the volume of the adipocytes, which, during the subsequent application of the treatment according to steps c) and d), enables a maximum of cells to be treated in the volume of fatty tissue accessible to those treatments.

Step c) can be implemented approximately one to two weeks, preferably one week, after the last administration of the “preparative” solution having an osmolarity of from 167 mOsm/l to 330 mOsm/l and comprising from 20 to 110 mOsm/l of potassium.

Advantageously, the solution used in step c) (destruction solution) may comprise a vasoconstrictor, an anaesthetizing agent, sodium chloride and/or bicarbonate. Preferably, this solution comprises 13.2 mOsm/l of xylocaine and/or 1.2 mOsm/l of adrenaline. This solution may also comprise from 1000 mg/l to 4800 mg/l of phosphatidylcholine, preferably from 1000 mg/l to 1200 mg/l, and optionally from 13.2 mg/l to 26.4 mg/l of tiratricol, preferably 13.2 mg/l.

An example of a destruction solution that can be used to implement step c) comprises 10 ml of 2% xylocaine, 1 ml of adrenaline at 1 mg and 2.5 ml of 10% NaCl or 4.2 ml of 8.4% sodium bicarbonate, in water, for an injectable preparation, in an amount sufficient to make up 250 ml.

In parallel with a reduction in volume, the preliminary treatment according to step a) brings about, in some cases, the appearance of a zone which is readily compressible and loose to the touch. Advantageously, the total osmolarity of the destruction solution used in step c) can be adjusted as a function of the “coefficient of compressibility” of the tissue. This coefficient of compressibility is defined as being the ratio of the thickness of the subcutaneous tissue measured by echography, without exerting pressure on the tissue, to the minimum thickness of the same tissue, as measured by the operator by compressing the tissue to the maximum extent with the echography probe. For example, when the coefficient of compressibility is from 1 to 1.5, a solution whose total osmolarity is from 90 to 75 mOsm/l may be used; for a coefficient of compressibility of from 1.5 to 2.5, a solution having an osmolarity of from 75 to 60 mOsm/l may advantageously be used; for a coefficient of compressibility greater than 2.5, a solution having an osmolarity of from 60 to 20 mOsm/l may advantageously be used.

A mechanical treatment capable of bringing about the destruction of the membrane of adipocytes present in the fatty tissue may consist in applying a transcutaneous ultrasound field to the fatty tissue, under conditions sufficient to obtain a destruction of adipocytes, and/or in massaging the fatty tissue by massage of the palpation-rolling type, for example.

The application of an ultrasound field to hyperhydrated adipocytes enables the adipocytes to be destroyed by cavitation. In general, the ultrasound treatment can be implemented immediately after the administration of the hypotonic destruction solution. It is possible to ascertain that the state of hydration of the adipocytes is sufficient by checking that the fatty tissue has an increased firmness compared with the firmness observed before the administration of the hypotonic solution of step c). The determination of the ultrasound operating conditions is within the competence of the person skilled in the art. Generally, a low frequency ultrasound apparatus is used, limiting the power emitted by the transducer to a mean of 3 watts/cm². Owing to this power limitation, two transducers having very similar resonant frequencies may be used to improve the amplitude of the destructive cavitation effect on the fatty tissue, as described in patent application FR2809018. The application of the ultrasound field generally lasts for from 3 to 6 minutes. It is possible to use an ultrasound apparatus of 1 mega Hz.

Advantageously, the method according to the invention also comprises the administration of a so-called “follow-up treatment” solution having an osmolarity of from 180 to 200 mOsm/l, preferably of approximately 200 mOsm/l. This solution can be administered after absorption of the fibrosis induced by the treatment of steps c) and d), that is to say, approximately one month after the implementation of steps c) and d). The administration of this follow-up treatment solution can be repeated. Generally, the follow-up treatment comprises three administrations, at intervals of approximately one month, of a follow-up treatment solution.

The determination of the volume of solution to be administered in accordance with steps a), b) and c) is within the competence of the person skilled in the art. These solutions are administered by transcutaneous injection. By way of example, it is possible to carry out an administration in accordance with a grid provided on the skin covering the fatty tissue treated in accordance with injection points distributed at 5-cm intervals on parallel lines which are themselves spaced by 3 cm. According to this method, the solutions can be administered at a level of 5 ml of solution per centimetre of fatty tissue thickness.

Advantageously, the preparation, destruction and follow-up treatment solutions are administered at a temperature of from 38 to 40° C.

1. Aqueous solution having an osmolarity of from 167 mOsm/l to 330 mOsm/l and comprising from 20 to 110 mOsm/l of potassium.

2. Aqueous solution according to claim 1, having an osmolarity of from 167 mOsm/l to 257 mOsm/l.

3. Aqueous solution according to claim 1 or 2, having an osmolarity of from 227 mOsm/l to 257 mOsm/l.

4. Aqueous solution according to any one of claims 1 to 3, comprising from 25 to 110 mOsm/l of potassium.

5. Aqueous solution according to any one of claims 1 to 4, comprising from 40 to 70 mOsm/l of potassium.

6. Aqueous solution according to any one of claims 1 to 5, comprising from 50 to 60 mOsm/l of potassium.

7. Aqueous solution according to any one of claims 1 to 6, also comprising 13.2 mOsm/l of xylocaine and/or 1.2 mOsm/l of adrenaline.

8. Aqueous solution according to any one of claims 1 to 7, also comprising from 1000 mg/l to 4800 mg/l of phosphatidylcholine and/or from 13.2 mg/l to 26.4 mg/l of tiratricol.

9. Aqueous solution having an osmolarity of from 20 mOsm/l to 330 mOsm/l and comprising from 1000 mg/l to 4800 mg/l of phosphatidylcholine and/or from 13.2 mg/l to 26.4 mg/l of tiratricol.

10. Aqueous solution according to claim 9, having an osmolarity of from 120 to 330 mOsm/l.

11. Aqueous solution according to claim 9, having an osmolarity of from 20 to 90 mOsm/l.

12. Use of an aqueous solution according to any one of claims 1 to 11, for the aesthetic treatment of cellulite.

13. Method for the aesthetic treatment of cellulite comprising the steps that consist in:
a) administering an aqueous solution having an osmolarity of from 167 mOsm/l to 330 mOsm/l and comprising from 20 to 110 mOsm/l of potassium, as defined in any one of claims 1 to 11, to fatty tissue;
b) optionally repeating step a) until the thickness of the fatty tissue, determined by echography, is less than or equal to 2 cm, especially from 1 to 2 cm;
c) administering an aqueous solution having an osmolarity of from 20 mOsm/l to 90 mOsm/l to the fatty tissue;
d) applying to the said tissue a mechanical treatment capable of bringing about the destruction of the membrane of adipocytes present in the fatty tissue; and
e) optionally administering a solution having an osmolarity of from 180 to 200 mOsm/l to the fatty tissue treated by the mechanical treatment.
